(19) United States
(12) Patent Application Publication
(10) Pub. No.: US 2005/0216082 A1
(43) Pub. Date: Sep. 29, 2005

(54) Threaded intervertebral implant

(75) Inventors: Jacque R. Wilson, Fort Wayne, IN (US); John F. Hoffman, Fort Wayne, IN (US); Adam F. Cox, Fort Wayne, IN (US)

Correspondence Address: FAEGRE & BENSON;ATTN: PATENT DOCKETING, 2200 WELLS FARGO CENTER, 90 SOUTH 7TH STREET, MINNEAPOLIS, MN, 55402-3901, US

(73) Assignee: Zimmer Spine, Inc., Minneapolis, MN (US)

(21) Appl. No.: 11/088,642

(22) Filed: Mar. 24, 2005

Related U.S. Application Data
(60) Provisional application No. 60/556,337, filed on Mar. 25, 2004.

(51) Int. Cl. A61F 2/44

(52) U.S. Cl. 623/17.11

(57) ABSTRACT

A threaded intervertebral implant or cage which includes a cylindrical or tapered body made from a biocompatible metal such as titanium, or a radiolucent plastic material. The cage includes a pair of opposing, threaded arcuate contact portions oriented in the superior/inferior direction for threading, supporting engagement with the endplates of adjacent vertebrae when the cage is implanted. Additionally, the cage includes a threaded lateral side having an arcuate profile, and a medial side having a concave channel extending along the length of the cage. The channel allows two cages to be implanted in a side-by-side manner between adjacent vertebrae with the channel of a first implanted cage receiving the arcuate portions of an adjacent second cage while the second cage is implanted. In this manner, a pair of adjacent cages may be implanted very close to one another. Also, when two cages are implanted in a side-by-side manner, the concave channels of the medial sides of the cages face one another to define a substantially enclosed intercage space between the cages in which bone growth-promoting material may be packed to facilitate fusion between adjacent vertebrae.

### BACKGROUND OF THE INVENTION

1. Field of the Invention

The present invention relates to threaded intervertebral implants for stabilizing and promoting fusion between adjacent vertebral bodies in the human spinal column.

2. Description of the Related Art

Chronic back problems cause pain and disability for a large segment of the population. Frequently, the cause of back pain is traceable to diseased disk material between adjacent vertebrae. When the disk material is diseased, the adjacent vertebrae may be inadequately supported, resulting in persistent pain.

Surgical techniques have been developed to remove the diseased disk material and fuse the joint between adjacent vertebrae. Stabilization and/or arthrodesis of the intervertebral joint can reduce the pain associated with movement of an intervertebral joint having diseased disk material. Generally, vertebral fusion techniques involve removal of the diseased disk and packing the void area with a suitable matrix for facilitating a bony union between the adjacent vertebrae.

One well-known fusion technique currently in use involves the use of threaded intervertebral implants, commonly referred to as “cages”, which are threadably inserted between adjacent vertebral bodies of the spinal column after the diseased disk is removed. Typically, a pair of cages are inserted in a side-by-side manner between adjacent vertebrae, with the threads of the cages threadedly engaging the hard cortical bone of the adjacent vertebral endplates. After the cages are implanted, the cages support and take up the load between the adjacent vertebrae. Additionally, the cages may include one or more openings therein to facilitate the growth of bone between the vertebrae and through the cages to promote fusion of the adjacent vertebral bodies. Bone growth-promoting material, such as bone graft material or a suitable synthetic biocompatible bone growth-promoting material, may be packed into the cages either before or after implantation of the cages to promote bony ingrowth into and through the cages.

When a pair of cages are implanted in a side-by-side manner between adjacent vertebrae, the cylindrical profile of the cages necessitates that the cages have a combined width which is equal to at least the combined diameters of the cages. In this manner, it can be difficult to place a pair of cages close to one another while insuring that the threads of the cages engage the vertebral endplates to a sufficient depth, and the combined width of the cages may be too wide for each of the cages to fit between the vertebrae without projecting beyond the intradiscal space between the adjacent vertebrae.

Additionally, it is known to manufacture cages from radio-opaque biocompatible metals, such as titanium, which have a very high load strength, as well radiolucent plastic materials. Use of radiolucent plastic materials advantageously allows the cages to be substantially “invisible” in an x-ray image, such that bony ingrowth and fusion of adjacent vertebrae can be more easily viewed after implantation of the cages. However, radiolucent plastic materials may have somewhat less load-bearing capability than biocompatible metals such as titanium.

What is needed is a threaded intervertebral implant which is an improvement over the foregoing.

### SUMMARY OF THE INVENTION

The present invention provides a threaded intervertebral implant or cage which includes a cylindrical or tapered body made from a biocompatible metal such as titanium, or a radiolucent plastic material. The cage includes a pair of opposing, threaded arcuate contact portions oriented in the superior/inferior direction for threading, supporting engagement with the endplates of adjacent vertebrae when the cage is implanted. Additionally, the cage includes a threaded lateral side having an arcuate profile, and a medial side having a concave channel extending along the length of the cage. The channel allows two cages to be implanted in a side-by-side manner between adjacent vertebrae with the channel of a first implanted cage receiving the arcuate portions of an adjacent second cage while the second cage is implanted. In this manner, a pair of adjacent cages may be implanted very close to one another. Also, when two cages are implanted in a side-by-side manner, the concave channels of the medial sides of the cages face one another to define a substantially enclosed intercage space between the cages in which bone growth-promoting material may be packed to facilitate fusion between adjacent vertebrae.

Additionally, an implant driver is provided, which includes a threaded finger which is received within the channel in the medial side of the cage to complete the thread around the entire outer periphery of the cage for easy threading insertion of the cage between adjacent vertebrae. The driver also includes a threaded end receivable within a threaded bore in the proximal end of the cage to securely attach the cage to the driver.

In one form thereof, the present invention provides a pair of intervertebral spinal implants for insertion within a disc space between adjacent vertebral bodies of a spine, each implant including a generally threaded body having a proximal end, a distal end, and a length therebetween, the body including opposing arcuate contact portions, and an arcuate lateral portion and a medial portion between the contact portions, the medial portion including a concave channel interrupting the thread and extending along the length of the body; whereby the channels of the medial portions of the implants face one another to define an open cavity between the implants when the implants are implanted adjacent one another between adjacent vertebral bodies.

In another form thereof, the present invention provides an intervertebral spinal implant for insertion within a disc space between adjacent vertebral bodies of a spine, including a generally cylindrical threaded body having a proximal end, a distal end, and a length therebetween, the body further including opposing arcuate contact portions, and a lateral and a medial portion between the contact portions, the lateral portion being arcuate with the thread substantially uninterrupted thereon, and the medial portion including a concave channel interrupting the thread and extending along the length of the body.

In a further form thereof, the present invention provides an intervertebral spinal implant for insertion within a disc space between adjacent vertebral bodies of a spine, including a body having a proximal end and a distal end and a length therebetween, the body further including a threaded leading portion at the distal end having a generally tapered profile, the leading portion including opposing arcuate contact portions and opposing lateral and medial portions between the contact portions; and a non-threaded trailing portion at the proximal end, the trailing portion having a generally cylindrical profile.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a perspective view of a cage according to a first embodiment of the present invention;

FIG. 2A is an anterior or posterior view showing the implantation of a pair of cages of FIG. 1 between adjacent vertebrae in which a first such cage, shown to the left, has been implanted and a second such cage, shown to the right, is being threadingly implanted adjacent the first cage;

FIG. 2B is an anterior or posterior view showing the cages of FIG. 2A implanted between adjacent vertebrae, with the channels in the medial sides of the cages facing one another to define an intercage space containing a bone growth-promoting material;

FIG. 3 is a perspective view of a driver according to the present invention for use in implanting the cage of FIG. 1;

FIG. 4 is a perspective view of a cage according to a second embodiment of the present invention; and

FIG. 5 is a lateral view showing one of the cages of FIG. 4 implanted between adjacent vertebrae, wherein another such cage (not visible) is disposed adjacent and behind the shown cage.

Corresponding reference characters indicate corresponding parts throughout the several views. The exemplifications set out herein illustrate preferred embodiments of the invention, and such exemplifications are not to be construed as limiting the scope of the invention any manner.

### DETAILED DESCRIPTION

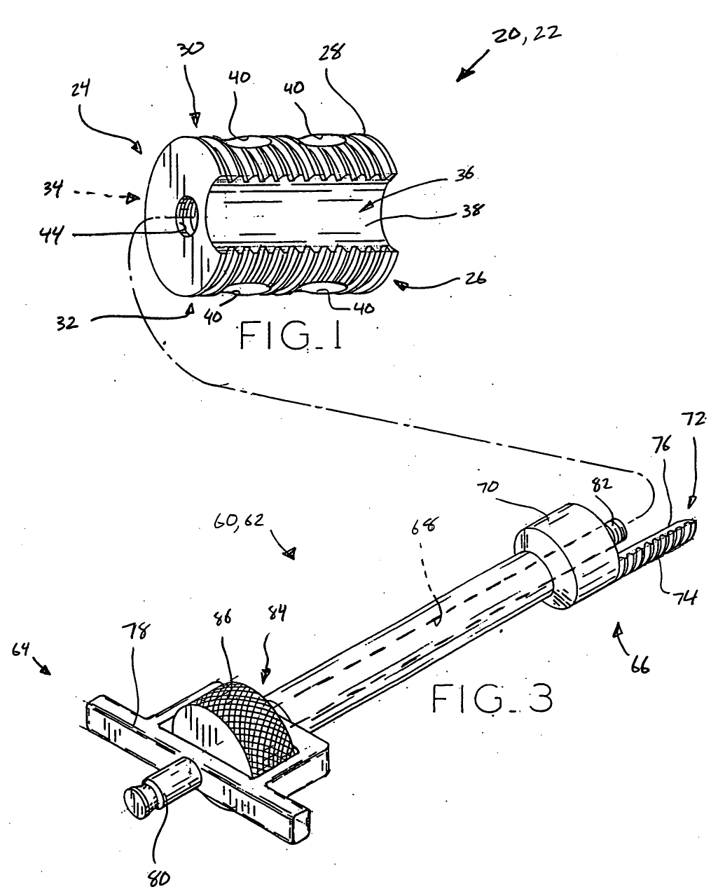

Referring to FIG. 1, an intervertebral implant or cage 20 in accordance with the present invention is shown, which includes a generally cylindrical body 22 having proximal end 24 and distal end 26. Cage 20 also includes a helical thread 28 therearound from distal end 26 to proximal end 24. Thread 28 may have a triangular or rectangular profile in cross-section, or a combination of the foregoing as desired. In one embodiment, thread 28 may have a sharp leading edge at distal end 26 which is configured to tap into bone, and thread 28 may also have a triangular profile in cross-section at distal end 26 to facilitate tapping, which gradually transitions to a rectangular profile in cross-section toward proximal end 24 for rigid engagement with bone and to reduce the potential of subsidence and retropulsion of cage 20.

Cage 20 also includes first and second opposing, threaded arcuate contact portions or sides 30 and 32 which are oriented in the superior/inferior direction when cage 20 is implanted, as described below. Cage 20 also includes a threaded arcuate lateral portion or side 34, and a medial portion or side 36 disposed between contact portions 30 and 32. Medial portion 36 is interrupted by a concave channel 38 which extends along the entire length of cage 20 from proximal end 24 to distal end 26 thereof. As described below, channel 38 allows two cages 20 to be implanted between adjacent vertebrae in a side-by-side manner close to one another.

Cage 20 may be made of a biocompatible metal, such as titanium, or alternatively, may be made of a radiolucent plastic material, such as polyether ether ketone (“PEEK”) with carbon fibers. One such material includes PEEK and carbon fibers in a weight ratio of 65% wt. PEEK to 35% wt. carbon fibers. When cage 20 is made of titanium or another metal, cage 20 is radio-opaque, such that cage 20 may easily be viewed in an x-ray image. However, when cage 20 is made of a radiolucent material such as PEEK/carbon fiber, cage 20 does not appear in an x-ray image. Optionally, when cage 20 is made of a radiolucent material, cage 20 may include radio-opaque markers embedded therewithin, such as tantalum beads, for example, with the radio-opaque markers placed near proximal and distal ends 24 and 26 of cage 20 in a desired pattern or arrangement such that the rotational position of cage 20 may be easily determined by viewing an x-ray image of cage 20.

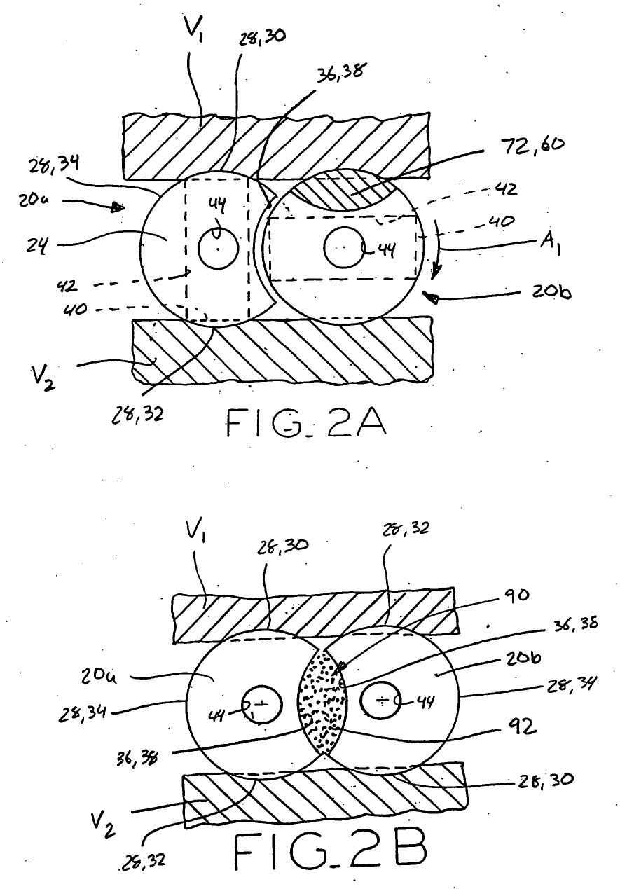

Cage 20 may have a substantially open interior, including bone ingrowth openings 40 in first and second contact portions 30 and 32 of cage 20 which are in communication with the open interior of cage 20. Typically, cage 20 may include a more completely open, hollow interior when cage 20 is made of a metal such as titanium, which has a relatively strong load-bearing capacity. However, when cage 20 is made of a radiolucent plastic such as PEEK/carbon fiber, the interior of cage 20 may not be as completely open, as the lesser load-bearing capacity of the radiolucent plastic may necessitate that cage 20 have thicker walls in one or more of its contact portions 30 and 32, lateral portion 34, and medial portion 36, such that body 22 of cage 20 is relatively solid. In this embodiment, cage 20 may include bone ingrowth openings 40 in the opposing contact portions 30 and 32 which are connected by through bores 42 which extend through body 22 of cage 20 as shown in FIG. 2A. Before cage 20 is implanted, bone growth-promoting material such as grafts or synthetic materials may be packed within the open interior and/or through bores 42 of cage 20.

However, regardless of whether cage 20 includes a relatively open, hollow interior within body 22 or a more solid body 22 with through bores 42, openings 40 are disposed in contact portions 30 and 32 of cage 20, with multiple openings 40 oriented along the longitudinal axis of cage 20, as shown in FIG. 1. In this manner, openings 40 are in contact with the endplates of adjacent vertebrae upon implantation, as discussed below. Although cage 20 is shown including a pair of bone ingrowth openings 40 in each of contact portion 30 and 32, the number and size of bone ingrowth openings 40 may vary, and bone ingrowth openings 40 may be formed as circular openings, or alternatively may be formed as ovals, radiused squares or rectangles, or elongated slots as desired. Generally, bone ingrowth openings 40 are sized as large as possible to promote bony ingrowth into and through cage 20, while still maintaining the structural and load-bearing capacity of cage 20.

Distal end 26 of cage 20 may be generally flat, or may be rounded or bullet-shaped as desired to facilitate insertion of cage 20 between adjacent vertebrae during implantation. Proximal end 24 of cage 20 is generally flat, and includes threaded bore 44 therein for attaching cage 20 to driver 60, as described below.

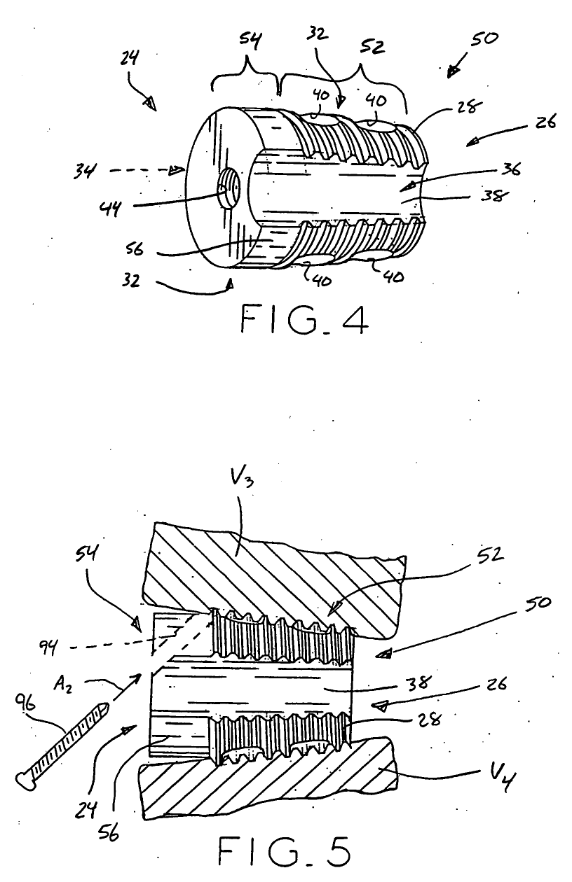

Referring to FIG. 4, threaded intervertebral implant or cage 50 according to a second embodiment is shown, which includes many features which are similar or identical to cage 20 of the first embodiment described above, and identical reference numerals have been used to denote identical or substantially identical features between cages 20 and 50. Cage 50 includes body 22 with a first or leading portion 52 at distal or posterior end 26 and a second or trailing portion 54 at proximal or anterior end 24. Leading portion 52 includes thread 28, and has a conical-shaped or tapered profile, with a smaller diameter at distal end 26 which gradually increases toward proximal end 24 and trailing portion 54 of cage 50. Normally, the angle of taper of first portion 52 of cage 50 is between 7° and 9°, which corresponds to the normal lordotic angle between adjacent vertebrae in the lumbar area of the human spine. However, the angle of taper of first portion 52 of cage 20 may vary as needed.

Second portion 54 of cage 50 is generally cylindrical in shape, including annular outer surface 56, and lacks thread 28. Second portion 54 of cage 50 may be solid, as opposed to having a hollow or open interior, to enhance the load-bearing strength of cage 50. Although second portion 54 of cage 50 is shown on the proximal or anterior end 24 of cage 50 in FIGS. 4 and 5, second portion 54 of cage 50 may also be on the distal or posterior end 26 of cage 50. In this manner, the distal end 26 of cage 50 would include a cylindrical portion extending therefrom which, when cage 50 is implanted, would be disposed between the posterior portions of adjacent vertebrae.

Similar to cage 20 described above, cage 50 may be made of radio-opaque or radiolucent materials, and first portion 52 of cage 50 additionally includes arcuate contact portions 30 and 32, arcuate lateral side 34, medial side 36 with convex channel 38, bone ingrowth openings 40, and second portion 54 of cage 50 includes threaded bore 44.

Referring to FIG. 3, a driver 60 is shown for use in implanting cages 20 and 50 in the manner described below. Driver 60 generally includes body 62 having proximal end 64 and distal end 66, and a hollow interior bore therethrough through which shaft 68 is rotatably disposed. Body 62 additionally includes attachment hub 70 having finger 72 extending therefrom. Finger 72 has a convex, threaded first side 74, and has an opposing, non-threaded convex side 76 which conforms to the concavity of channel 38 of cages 20 and 50. For use with tapered cage 50, the first, threaded side 74 of finger 72 of driver 60 may be tapered in shape. Proximal end 64 of driver 60 includes handle 78 which may be grasped by a surgeon to rotate driver 60 and a cage 20 or 50 which is attached to driver 60 as described below. Additionally, handle 78 includes a Hudson coupling 80 for attachment of other structures such as an auxiliary handle or slap hammer (note shown) to driver 60. Shaft 68 includes threaded distal end 82 for engagement within threaded bore 44 of cages 20 and 50, and includes proximal end 84 with thumbwheel 86.

Further details regarding the surgical instrumentation and methods used for preparing the interdiscal space between adjacent vertebrae, and the surgical instrumentation and methods for preparing insertion bores for the placement of cages 20 and 50 between adjacent vertebrae are discussed in U.S. Pat. Nos. 5,458,638, 5,489,308, 5,865,847, 5,897,593, 6,156,040, 6,165,219, and 6,224,631, the disclosures of which are expressly incorporated herein by reference. Generally, after the interdiscal space has been prepared, a pair of adjacent circular bores are drilled between adjacent vertebrae as described in the above-identified patents, followed by reaming and tapping the bores. For tapered cage 50, special tapered drills, reamers, and taps may be used which have a profile corresponding to the lordotic taper angle of cage 50, as described in the above-incorporated U.S. Pat. Nos. 5,865,847, 5,897,593, and 6,165,219. Generally, cage 20 may be implanted according to anterior, posterior, oblique or translateral surgical procedures, while tapered cage 50 is best implanted according to an anterior surgical procedure.

Cage 20 is attached to driver 60 by first inserting finger 72 of driver 60 into channel 38 of cage 20 from proximal end 24 toward distal end 26 of cage 20. Thereafter, distal end 82 of shaft 68 of driver 60 is threaded into threaded bore 44 in proximal end 24 of cage 20 to securely attach cage 20 to driver 60, with proximal end 24 of cage 20 in abutment with attachment hub 70 of driver 60. Additionally, attachment hub 70 of driver 60 and proximal end 24 of cage 20 may include interfitting protrusions and recesses (not shown) to enhance transfer of rotational torque from driver 60 to cage 20. When cage 20 is attached to driver 60, the threads on side 74 of finger 72 align and conform with threads 28 of cage 20 to thereby complete thread 28 of cage 20 around the entire outer periphery of cage 20.

Thereafter, referring to FIG. 2A, driver 60 is used by a surgeon to rotationally thread a first cage 20a into a tapped circular bore between adjacent vertebrae V₁ and V₂ which is disposed to the left in FIG. 2A. Upon completion of threading the first cage 20a between adjacent vertebrae V₁ and V₂, medial side 36 of cage 20a with channel 38 is oriented medially as shown in FIG. 2A. Thereafter, thumbwheel 86 of driver 60 is rotated to disengage distal end 82 of driver 60 from threaded bore 44 of cage 20, followed by sliding finger 72 of driver 60 outwardly from channel 38 of cage 20 to disengage driver 60 from cage 20.

Driver 60 is then attached to a second cage 20b in the same manner described above and, as shown in FIG. 2A, second cage 20b is rotationally threaded into a second circular bore adjacent first cage 20a as denoted by arrow A₁. While threading second cage 20b into its circular bore adjacent the first cage 20a, the arcuate outer periphery of second cage 20b is received within channel 38 of first cage 20a. In this manner, first and second cages 20a and 20b may be implanted in a side-by-side manner very close to one another, with the combined width of the first and second cages 20a and 20b less than the combined diameters of the cages, such that cages 20a and 20b together will have an overall reduced width profile as compared to known completely cylindrical cages which are inserted in a side-by-side manner adjacent to one another as with known surgical techniques.

Referring to FIG. 2B, medial side 36 and channel 38 of second cage 20b is aligned with medial side 34 and channel 38 of first cage 20a upon completion of implantation of second cage 20b to define a substantially enclosed intercage space or cavity 90 between first and second cages 20a and 20b. Advantageously, after implanting cages 20a and 20b, cavity 90 may be filled with bone graft material, such as autograft, allograft, or zenograft bone material, or a synthetic bone growth-inducing material such as porous metallics, ceramics, polymers, or composite materials, for example, calcium hydroxyapatite or a trabecular metal such as Hedrocel® (, available from Implex Corp. of Allendale, N.J., or Trabecular Metal™, available from Zimmer, Inc. of Warsaw, Ind. to promote bony ingrowth into cavity 90 to enhance subsequent fusion of the adjacent vertebrae V₁ and V₂. Additionally, after cages 20a and 20b are implanted, bony ingrowth is facilitated through bone ingrowth openings 40 of cages 20a and 20b into the interior of cages 20a and 20b to promote fusion of the adjacent vertebrae V₁ and V₂.

Cages 50 are implanted in the same manner as that described above with respect to cages 20a and 20b. Referring to FIG. 5, it may be seen that the tapered first portion 52 of cage 50 conforms to the lordotic angle between adjacent vertebrae V₃ and V₄, with thread 28 engaging the endplates of the adjacent vertebrae V_{3 and V}₄. Second portion 54 of cage 50 may also provide additional support to the adjacent vertebrae V₃ and V₄. Further, second portion 54 of cage 50 may provide an interface for the use of supplementary bone screws (not shown) with cage 50. For example, as shown in FIG. 5, second portion 54 of cage 50 may be formed relatively solid, with one or more transverse bores 94 therethrough which are aligned at an oblique angle to the longitudinal axis of cage 50 for insertion of one or more bone screws 96 from proximal end 24 of cage 50 through the bores 94 along the direction of arrow A₂ and into the bone of vertebrae V₃ and V₄ to further secure the implanted position of cage 50 with respect to V₃ and V₄.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A pair of intervertebral spinal implants for insertion within a disc space between adjacent vertebral bodies of a spine, each implant comprising:
a generally threaded body having a proximal end, a distal end, and a length therebetween, said body including opposing arcuate contact portions, and an arcuate lateral portion and a medial portion between said contact portions, said medial portion including a concave channel interrupting said thread and extending along said length of said body;
whereby said channels of said medial portions of said implants face one another to define an open cavity between said implants when said implants are implanted adjacent one another between adjacent vertebral bodies.

2. The implants of claim 1, wherein said body of each said implant has a generally cylindrical profile along said length.

3. The implants of claim 1, wherein said body of each said implant is tapered along at least a portion of said length.

4. The implants of claim 1, wherein said implants each include an interior cavity, and said contact portions each include at least one bone ingrowth opening interrupting said threads and communicating with said interior cavity.

5. The implants of claim 1, wherein said body of each implant is generally solid, and further includes at least one bone ingrowth opening extending through said body between said opposing arcuate contact portions.

6. The implants of claim 5, wherein said implants are each made of a radiolucent plastic material.

7. An intervertebral spinal implant for insertion within a disc space between adjacent vertebral bodies of a spine, comprising:
a generally cylindrical threaded body having a proximal end, a distal end, and a length therebetween, said body further including opposing arcuate contact portions, and a lateral and a medial portion between said contact portions, said lateral portion being arcuate with said thread substantially uninterrupted thereon, and said medial portion including a concave channel interrupting said thread and extending along said length of said body.

8. The implant of claim 7, wherein said implant includes an interior cavity, and said contact portions each include at least one bone ingrowth opening interrupting said threads and communicating with said interior cavity.

9. The implant of claim 7, wherein said body of said implant is generally solid, and further includes at least one bone ingrowth opening extending through said body between said opposing arcuate contact portions.

10. The implant of claim 9, wherein said implant is made of a radiolucent plastic material.

11. An intervertebral spinal implant for insertion within a disc space between adjacent vertebral bodies of a spine, comprising:
a body having a proximal end and a distal end and a length therebetween, said body further comprising:
a threaded leading portion at said distal end having a generally tapered profile, said leading portion including opposing arcuate contact portions and opposing lateral and medial portions between said contact portions; and
a non-threaded trailing portion at said proximal end, said trailing portion having a generally cylindrical profile.

12. The implant of claim 11, further comprising a concave channel extending along the length of said body through said leading and trailing portions, said channel interrupting said thread along said medial portion.

13. The implant of claim 11, wherein said implant includes an interior cavity, and said contact portions each include at least one bone ingrowth opening interrupting said threads and communicating with said interior cavity.

14. The implant of claim 11, wherein said body of said implant is generally solid, and further includes at least one bone ingrowth opening extending through said body between said opposing arcuate contact portions.

15. The implant of claim 14, wherein said implant is made of a radiolucent plastic material.
